(19) United States
(12) Patent Application Publication
(10) Pub. No.: US 2026/0306376 A1
(43) Pub. Date: Oct. 8, 2026

(54) HAIR FIBER STRENGTHENING COMPOSITIONS

(71) Applicant: ELC MANAGEMENT LLC, Melville, NY (US)

(72) Inventor: Sara CARLSON, Ham Lake, MN (US)

(21) Appl. No.: 19/461,673

(22) Filed: Jan. 27, 2026

Related U.S. Application Data
(60) Provisional application No. 63/751,797, filed on Jan. 30, 2025.

(51) Int. Cl. A61K 8/42; A61K 8/31; A61K 8/34; A61K 8/41; A61Q 5/12

(52) U.S. Cl. CPC A61K 8/42; A61K 8/31; A61K 8/345; A61K 8/416; A61Q 5/12; A61K 2800/33

(57) ABSTRACT

Haircare compositions containing at least one hair fiber strengthening active ingredient, at least one humectant, at least one emollient, at least one conditioner, and water provide a hair fiber strengthening benefit when applied to hair. The haircare composition may be applied as a pre-shampoo in a haircare routine to decrease chemical and mechanical stresses on hair fibers during the shampooing process.

### FIELD OF THE INVENTION

The present invention pertains to hair fiber strengthening compositions, more specifically to pre-shampoo compositions that promote the strengthening of human hair fibers.

### BACKGROUND OF THE INVENTION

Having a full, healthy and manageable head of hair is a common concern of men and women. Other than the number of hair fibers on a person's head, a full head of natural, healthy, manageable hair depends on the diameter and cross-sectional area of the hair fibers, as well as various mechanical properties thereof.

There are several factors that should be considered when formulating haircare compositions; not only consumer needs such as cleansing, conditioning, and styling, but also effectively strengthening hair fibers. Hair is composed primarily of keratin, a protein that provides structure and strength. However, hair fibers are subject to daily chemical, mechanical, and thermal stresses that degrade keratin integrity and lead to weakened, brittle hair that is prone to breakage. Several common practices can have hair-damaging effects include repeated combing, frequent shampooing, heat styling, and chemical treatments such as dyeing or perming. The concerns are also particularly pronounced for those with fine hair. For the purposes of this description, we define fine hair diameter as less than about 65 μm, medium hair diameter as about 65 μm to about 90 μm, and coarse hair diameter as more than about 90 μm.

One particular concern mentioned is the potential for hair-damaging effects from shampooing. When applying shampoo and rinsing, there is a high likelihood for longer hair fibers to get tangled and knotted, resulting in breakage. Additionally, during the process of applying shampoo and rinsing, the water can cause cuticle layers to open and become waterlogged, which results in stretching and swelling of the hair fibers, which can also make the hair prone to breakage. Furthermore, traditional shampoos often contain surfactants that are effective at removing dirt and oils but may also strip away protective lipids and moisture from the hair fiber, increasing susceptibility to damage. Additionally, for some users, the water used typically during the shampoo process may be hard water, which contains heavy metals which may damage the hair.

While post-wash conditioners and treatments that restore some of this lost moisture and hair fiber strength are known in the field, these solutions do not fully mitigate the damage already incurred during the washing process. There has therefore been an increased interest in developing pre-shampoo compositions that prepare and protect the hair before exposure to chemical and mechanical stresses. However, it has been found that pre-shampoo compositions on the market do not provide sufficient hair strengthening, when compared to effective post-wash conditioners and treatments.

Pre-shampoo compositions are applied to the hair prior to shampooing and can form a protective layer around the hair fiber. This effect can help reduce moisture loss, minimize friction during cleansing, and mitigate the harsh effects of surfactants on the hair fiber's structural integrity. Additionally, pre-shampoo products can also deliver targeted benefits, such as nourishment, detangling, and strengthening, directly to the hair fibers before they are subjected to the cleansing process.

Despite their benefits, formulating an effective pre-shampoo composition for hair strengthening poses unique technical challenges. The composition must adhere effectively to the hair fiber, provide substantive conditioning and protective effects, and resist being washed away entirely during subsequent shampooing. On the other hand, the pre-shampoo should not create a film that prevents a subsequent shampoo from effectively performing its cleansing, such as removing dirt and oils. Furthermore, any active ingredients responsible for strengthening the hair must penetrate and interact with the hair fiber structure to improve its mechanical properties without leaving undesirable residues.

Therefore, there is a need for pre-shampoo compositions that addresses these formulation challenges by providing superior hair fiber protection and strengthening properties, while also maintaining compatibility with standard shampooing practices. The present invention offers a novel composition that enhances hair fiber strength and resilience and also reduces breakage, to ultimately promote healthier, stronger hair.

### SUMMARY OF THE INVENTION

Hair fiber strengthening compositions and corresponding methods of treating hair are provided herein.

The invention is directed to hair fiber strengthening compositions comprising (a) at least one hair fiber strengthening active ingredient, (b) at least one humectant, (c) at least one emollient, (d) at least one conditioner, and (e) water.

### DETAILED DESCRIPTION OF THE INVENTION

### Definitions and Conventions

The term, “comprising” means that a list of elements is not necessarily limited to those explicitly recited.

As used herein, “cosmetically acceptable” means that compositions or components are suitable for use in contact with human keratinous tissue.

The term “molecular weight” refers to the weight average molecular weight, unless otherwise stated.

The term “active ingredients” and “actives” are used interchangeably.

Unless otherwise stated: all numerical amounts are understood to be modified by the word “about,” all percentages are by weight of the total composition, and all ratios are weight ratios. It should be understood that every numerical range expressly recited herein will include every narrower numerical range that falls within such broader numerical range, as if such narrower numerical ranges were all expressly recited herein.

As used herein, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means.

As used herein, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

As used herein, “and/or” means “and” or “or”. For example, “A and/or B” means “A, B, or both A and B,” and “A, B, C, and/or D” means “A, B, C, D, or a combination thereof,” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

### Hair Fiber Strengthening Composition

Described herein are hair fiber strengthening compositions comprising:
at least one hair fiber strengthening active ingredient,
at least one humectant,
at least one emollient,
at least one conditioner, and
water.

In the present invention, the combination of the hair fiber strengthening active ingredient, humectant, emollient, conditioner, and water is believed to provide a synergistic effect that boosts the hair fiber strengthening properties. In particular, some specific ingredient combinations described below are suspected to have unexpected synergy on the hair fiber strengthening properties of the composition. Additional optional cosmetically acceptable components not described herein may also be included in the hair fiber strengthening composition. The hair fiber strengthening composition according to the invention is also useful for fine hair, medium hair, and coarse hair.

The hair fiber strengthening composition is in the form of an emulsion. Additionally, the hair thickening composition is preferably a pre-shampoo composition, to be applied in a haircare regiment before shampooing and optionally, conditioning. Using the hair fiber strengthening composition according to the invention as a pre-shampoo helps to reduce moisture loss, minimize friction during cleansing, and mitigate the harsh effects of surfactants in shampoos on the hair fiber's structural integrity.

### Hair Fiber Strengthening Active Ingredients

The hair fiber strengthening active ingredients are a key component in the present invention. The hair fiber strengthening active works by penetrating within the hair fiber to the cortex and creating additional hydrogen bonds and/or ionic bonds within the keratin structure. This allows the hair fiber to increase in strength, and reduces hair fiber breakages due to chemical, heat, and mechanical stresses.

One example of the hair fiber strengthening active is Fiberhance™ BM Solution, available from Ashland Inc., which is a mixture containing hydroxypropyl gluconamide and hydroxypropylammonium gluconate. Another example of the hair fiber strengthening active is TeraBond™, available from Actera Ingredients, Inc., which is a mixture containing Aspergillus ferment and arginine.

The present hair fiber strengthening composition contains the hair fiber strengthening active in an amount of at least 2% by weight, preferably 2% to 6% by weight, further preferably approximately 4% by weight, based on a total weight of the haircare formulation.

### Conditioner

Conditioners are a key component of haircare formulations, primarily used to improve the texture, smoothness, and manageability of hair. They are typically able to adhere to the surface of hair fibers, forming a protective film that smooths cuticles and reduces friction. In a pre-shampoo composition, conditioners play a critical role in reducing the mechanical stress encountered during shampooing by enhancing the hair's slip and minimizing tangling.

In the present invention, the conditioner serves a dual function: it conditions the hair shaft and acts as a carrier for active strengthening agents. By forming a protective layer, the conditioner minimizes direct exposure of the hair's cortex to harsh cleansing agents, thereby preserving fiber integrity. Some non-limiting examples of suitable conditioning agents include behentrimonium chloride, cetrimonium chloride, or guar hydroxypropyltrimonium chloride, which are known for their detangling and smoothing properties.

### Emollient

Emollients are substances that soften and smooth the hair surface by filling in gaps and cracks along the cuticle layer. Common emollients used in haircare include natural oils, silicones, and fatty alcohols. In a pre-shampoo formulation, emollients can enhance moisture retention and create a barrier that protects against surfactant-induced dehydration. In this invention, the emollient works synergistically with other components to provide a lubricating effect that further reduces friction and breakage during shampooing. Some non-limiting examples of the emollient include mineral oils, plant-derived oils such as argan or jojoba oil, squalane, silicone oils, caprylic/capric triglyceride, and polyglyceryl-10 pentaoleate.

The present hair fiber strengthening composition contains emollients in an amount of 0.1% to 25% by weight, preferably 0.25% to 5% by weight, based on a total weight of the haircare formulation.

### Humectant

Humectants are hygroscopic substances that attract and retain moisture in the hair, preventing dehydration and reducing brittleness. Humectants can therefore enhance the flexibility of and reduce the risk of breakage of hair fibers. The inclusion of a humectant in a pre-shampoo composition in particular reduces the potential negative moisture stripping effects of shampooing. Some non-limiting examples of humectants include glycerin, butylene glycol, propylene glycol, and sorbitol.

The present hair fiber strengthening composition contains humectants in an amount of 1% to 30% by weight, preferably 10% to 30% by weight, and further preferably 10% to 25% by weight, based on a total weight of the haircare formulation. It has surprisingly been found that a level of humectants higher than 30% by weight results in a decrease in hair strengthening effects of the hair fiber strengthening composition.

### Water

The hair fiber strengthening composition according to the present invention is in the form of an emulsion, and therefore contains water as the primary solvent in the aqueous phase. The water serves as a carrier for other ingredients, facilitating the dispersion and uniform application thereof across the surface of the hair. In particular, water is necessary as a carrier for the bond-building active ingredient. Additionally, the water can provide a hydrating effect, allowing for the hair fibers to be more receptive to treatment.

The hair fiber strengthening composition therefore contains water in an amount of 40%-80% by weight, more preferably, from 50%-75% by weight, based on a total weight of the haircare formulation.

### Structurant

In some embodiments, the hair fiber strengthening composition includes a structurant to improve the viscosity, consistency, or texture of the emulsion. The suitability of a specific structurant depends on the specific components present in the aqueous phase and oil phase. In some preferred embodiments, the phase structurant is cetearyl alcohol, behenyl alcohol, or a mixture thereof. The hair fiber strengthening compositions may comprise at least one structurant in an amount of 0.10% to 4% by weight, preferably 1% to 3% by weight, based in each case on the total weight of the hair fiber strengthening composition.

### Additional Components

The hair fiber strengthening compositions may comprise additional optional cosmetically acceptable components. However, in general, any additional ingredient that negatively effects the hair strengthening effects, potency or effectiveness of active ingredients, or stability of the composition should be used sparingly or not at all. Non-limiting examples of additional cosmetic components include oils, waxes, fragrance oils, essential oils, moisturizers, humectants, thickeners, preservatives, emulsifiers, film formers, surfactants, stabilizers, and/or additional active ingredients.

### Method of Making Hair Fiber Strengthening Composition

The process for producing the hair fiber strengthening composition according to the present invention involves combining an aqueous phase and oil phase. The aqueous phase contains the water, hair fiber strengthening active ingredient, and humectant. The oil phase contains the conditioner and emollient. An optional alcoholic phase may also be mixed with the aqueous phase and oil phase. Additional optional cosmetically acceptable ingredients can also be included in the aqueous phase, the oil phase, the optional alcoholic phase, or can be added after the phases are combined. In particular, any temperature sensitive ingredients should be added after the phases are combined and the resulting mixture is cooled down.

### Method of Treating Hair

The hair fiber strengthening composition according to the invention can be used in a method of treating hair. The hair fiber strengthening composition should be applied to the hair and left to remain on the hair for at least 5 minutes, in order to see significant hair fiber strengthening benefits. In some cases, the hair fiber strengthening composition is left to remain on the hair for 10 minutes, 20 minutes, or even 30 minutes.

In a preferred embodiment, the hair fiber strengthening composition is a pre-shampoo. Accordingly, in some methods of treatment, after the hair fiber strengthening composition is applied to the hair and left to sit for a period of time as described above, the hair is then cleansed with a shampoo composition. A conditioner composition can optionally be applied thereafter.

### LIST OF EMBODIMENTS

The present invention also includes the following list of embodiments:
1. A haircare composition, comprising:
   at least one hair fiber strengthening active ingredient,
   at least one humectant,
   at least one emollient,
   at least one conditioner, and
   water.
2. The haircare composition according to embodiment 1, wherein the haircare composition is a pre-shampoo composition.
3. The haircare composition according to embodiments 1 or 2, wherein the at least one hair fiber strengthening active ingredient comprises hydroxypropylgluconamide and hydroxypropylammonium gluconate.
4. The haircare composition according to any of the preceding embodiments, wherein the at least one hair fiber strengthening active ingredient comprises Aspergillus ferment and arginine.
5. The haircare composition according to any of the preceding embodiments, wherein the haircare composition comprises the at least one hair fiber strengthening active ingredient in an amount of 2%-6% by weight, based on a total weight of the haircare composition.
6. The haircare composition according to any of the preceding embodiments, wherein the at least one humectant is butylene glycol, glycerin, or a mixture thereof.
7. The haircare composition according to any of the preceding embodiments, wherein the haircare composition contains the at least one humectant in an amount less than 30% by weight, based on a total weight of the haircare composition.
8. The haircare composition according to any of the preceding embodiments, wherein the at least one conditioner comprises behentrimonium chloride, hydroxypropyltrimonium chloride, polyacrylamidopropyl trimonium chloride, or a mixture thereof.
9. The haircare composition according to any of the preceding embodiments, wherein the at least one conditioner comprises behentrimonium chloride.
10. The haircare composition according to embodiment 7 or 8, wherein the haircare composition comprises the behentrimonium chloride in an amount of 0.75% by weight or more, based on a total weight of the haircare composition.
11. The haircare composition according to any of the preceding embodiments, wherein the haircare composition does not contain stearamidopropyl dimethylamine.
12. The haircare composition according to any of the preceding embodiments, wherein the at least one emollient comprises caprylic/capric triglyceride, polyglyceryl-10 pentaoleate, squalane, or a mixture thereof.
13. The haircare composition according to any of the preceding embodiments, wherein the at least one emollient comprises squalane.
14. The haircare composition according to any of embodiments 1-10, wherein the at least one emollient consists of squalane.
15. The haircare composition according to any of embodiments 11-13, wherein the haircare composition contains the squalane in an amount of 0.25% by weight, based on a total weight of the haircare composition.
16. The haircare composition according to any of the preceding embodiments, wherein the haircare composition further comprises at least one structurant.
17. The haircare composition according to embodiment 15, wherein the at least one structurant is cetearyl alcohol, behenyl alcohol, or a mixture thereof.
18. A method of treating hair, comprising:
   applying the haircare composition according to any of the preceding embodiments to the hair, and letting the haircare composition remain on the hair for at least 5 minutes.
19. The method of embodiment 17, further comprising:
   applying a shampoo composition to the hair and rinsing the hair thereafter.
20. The method of embodiment 18, further comprising:
   applying a conditioner composition to the hair and rinsing the hair thereafter.

### EXAMPLES

### Hair Fiber Strengthening Composition Examples

The following Tables 1 & 2 detail the ingredients of composition examples 1-12 according to the invention. All ingredient amounts are shown in their weight percentages, relative to the total weight of the corresponding composition.

TABLE 1
Hair Fiber Strengthening Composition Examples 1-6

| | | Ex. 1 | Ex. 2 | Ex. 3 | Ex. 4 | Ex. 5 | Ex. 6 |
|---|---|---|---|---|---|---|---|
| Hair Fiber | Fiberhance ™ BM Solution ¹ | 2.03 | 2.03 | 2.03 | 4.04 | 4.03 | 4.04 |
| Strengthening | TeraBond ™ ² | | | | | 2.02 | |
| Active | | | | | | | |
| Humectants | Butylene Glycol | 5.08 | 10.16 | 5.08 | 10.09 | 10.08 | 3.03 |
| | Glycerin | 5.08 | | 5.08 | 10.09 | 10.08 | |
| Water | | 77.05 | 76.54 | 76.03 | 64.08 | 62.10 | 64.08 |
| Conditioners | Behentrimonium Chloride | 0.39 | 0.39 | 0.39 | 0.77 | 0.77 | 0.77 |
| | Guar | 1.52 | 1.52 | 1.52 | 0.50 | 0.50 | 0.50 |
| | Hydroxypropyltrimonium | | | | | | |
| | Chloride | | | | | | |
| | Polyacrylamidopropyl | 0.51 | 0.51 | 0.51 | 0.50 | 0.50 | 0.50 |
| | Trimonium Chloride | | | | | | |
| | Stearamidopropyl | 1.52 | 1.52 | 1.52 | | | |
| | Dimethylamine | | | | | | |
| | Polyester-11 | | | 1.02 | | | |
| Structurants | Cetearyl Alcohol | 1.14 | 2.41 | 1.14 | 2.26 | 2.26 | 2.26 |
| | Behenyl Alcohol | 0.76 | | 0.76 | 0.76 | 0.76 | 0.76 |
| Emollients | Caprylic/Capric Triglyceride | 1.02 | 1.02 | 1.02 | 3.03 | 3.02 | 20.18 |
| | Polyglyceryl-10 Pentaoleate | 1.52 | 1.52 | 1.52 | 1.51 | 1.51 | 1.51 |
| | Squalane | 0.25 | 0.25 | 0.25 | 0.25 | 0.25 | 0.25 |
| Additional | Octyldodecyl Citrate | 0.51 | 0.51 | 0.51 | 0.50 | 0.50 | 0.50 |
| Ingredients | Crosspolymer | | | | | | |
| | Preservatives ³ | 1.32 | 1.32 | 1.32 | 1.31 | 1.31 | 1.31 |
| | Additional Active | 0.30 | 0.30 | 0.30 | 0.30 | 0.30 | 0.30 |
| | Ingredient Oils ⁴ | | | | | | |

¹ Fiberhance ™ BM Solution, available from Ashland Inc., is a mixture containing hydroxypropyl gluconamide and hydroxypropylammonium gluconate.
² TeraBond ™, available from Actera Ingredients, Inc., is a mixture containing Aspergillus ferment and arginine.
³ The preservative mixture is a combination of potassium sorbate, ethylhexylglycerin, tocopherol, benzyl alcohol, and Helianthus Annuus seed oil.
⁴ The additional active ingredient oils are a combination of Persea Gratissima oil, Camellia Oleifera seed oil, and Plukenetia Volubilis seed oil.

TABLE 2
Hair Fiber Strengthening Composition Examples 7-12

| | | Ex. 7 | Ex. 8 | Ex. 9 | Ex. 10 | Ex. 11 | Ex. 12 |
|---|---|---|---|---|---|---|---|
| Hair Fiber | Fiberhance ™ BM Solution | 4.04 | 4.03 | 4.03 | | | 4.03 |
| Strengthening | | | | | | | |
| Active | | | | | | | |
| Humectants | Butylene Glycol | 10.09 | 10.08 | 10.08 | 10.08 | 10.01 | 15.12 |
| | Glycerin | 10.09 | 10.08 | 10.08 | 10.08 | 10.01 | 15.12 |
| Water | | 46.92 | 65.88 | 68.65 | 68.15 | 69.40 | 54.03 |
| Conditioners | Behentrimonium Chloride | 0.77 | 0.38 | 0.77 | 0.77 | 0.77 | 0.77 |
| | Guar | 0.50 | 0.25 | 0.50 | 0.50 | 0.50 | 0.50 |
| | Hydroxypropyltrimonium | | | | | | |
| | Chloride | | | | | | |
| | Polyacrylamidopropyl | 0.50 | 0.50 | 0.50 | 0.50 | 0.50 | 0.50 |
| | Trimonium Chloride | | | | | | |
| Structurants | Cetearyl Alcohol | 1.14 | 2.41 | 1.14 | 2.26 | 2.26 | 2.26 |
| | Behenyl Alcohol | 0.76 | | 0.76 | 0.76 | 0.76 | 0.76 |
| Emollients | Caprylic/Capric Triglyceride | 20.18 | 3.02 | | 3.02 | 3.00 | 3.02 |
| | Polyglyceryl-10 Pentaoleate | 1.51 | 1.51 | | 1.51 | 1.50 | 1.51 |
| | Squalane | 0.25 | 0.25 | 0.25 | 0.25 | | 0.25 |
| Additional | Octyldodecyl Citrate | 0.50 | 0.50 | 0.50 | 0.50 | | 0.50 |
| Ingredients | Crosspolymer | | | | | | |
| | Preservatives | 1.31 | 1.31 | 1.31 | 1.31 | 1.31 | 1.31 |
| | Additional Active | 0.30 | 0.30 | 0.30 | 0.30 | | 0.30 |
| | Ingredient Oils | | | | | | |

In addition, three comparative examples were also tested, based on various haircare products on the market which purport to strengthen hair.

Comparative Example 1 is a marketed product named Fable & Mane HoliRoots™ Pre-Wash Strengthening Treatment Oil, and has the following ingredients:
Caprylic/Capric Triglyceride, Carthamus Tinctorius (Safflower) Seed Oil, Parfum (Fragrance), Sesamum Indicum (Sesame) Seed Oil, Withania Somnifera Root Extract, Phyllanthus Emblica Fruit Extract, Bacopa Monnieri Leaf Extract, Eclipta Prostrata Extract, Simmondsia Chinensis (Jojoba) Seed Oil, Ricinus Communis (Castor) Seed Oil, Uraria Picta Root Extract, Tribulus Terrestris Root Extract, Stereospermum Suaveolens Root Extract, Solanum Xanthocarpum Root Extract, Solanum Indicum Root Extract, Premna Serratifolia Root Extract, Glycyrrhiza Glabra (Licorice) Root Extract, Beta-Sitosterol, Gmelina Arborea Root Extract, Desmodium Gangeticum Root Extract, Aegle Marmelos Root Extract, Squalene, Olea Europaea (Olive) Fruit Oil, Vitis Vinifera (Grape) Seed Oil, Tocopherol, Phenoxyethanol, Benzyl Salicylate, Isoeugenol, Linalool.

Comparative Example 2 is a marketed product named Philip Kingsley Bond Builder Restructuring Pre-Shampoo Treatment, and has the following ingredients:
Aqua (water), butylene glycol, ethylhexyl palmitate, cetearyl alcohol, glyceryl stearate SE, panthenol, phenoxyethanol, bis-PCA dimethicone, hydroxyethyl acrylate/sodium acryloyldimethyl taurate copolymer, hydroxypropyl guar hydroxypropyltrimonium chloride, benzyl alcohol, squalane, parfum (fragrance), sodium stearoyl glutamate, aminomethyl propanol, limonene, disodium EDTA, disodium PEG-12 dimethicone sulfosuccinate, polysorbate 60, citric acid, dehydroacetic acid, sorbitan isostearate, linalool, citronellol, citral.

Comparative Example 3 is a marketed product named Joico HydraSplash Hydrating Conditioner, and has the following ingredients:
Water/Aqua/Eau, Cetearyl Alcohol, Behentrimonium Chloride, Isopropyl Palmitate, Stearyl Alcohol, Propylene Glycol, Phenoxyethanol, Ethylhexyl Olivate, Isopropyl Alcohol, Dipropylene Glycol, Fragrance/Parfum, Cocos Nucifera (Coconut) Oil, Cetyl Esters, Hydroxyethylcellulose, Bisamino PEG/PPG-41/3 Aminoethyl PG-Propyl Dimethicone, Helianthus Annuus (Sunflower) Seed Oil, Carthamus Tinctorius (Safflower) Seed Oil, Guar Hydroxypropyltrimonium Chloride, Polyquaternium-37, Bis-Ethyl (Isostearylimidazoline) Isostearamide, Tetrasodium Glutamate Diacetate, Propylene Glycol Dicaprylate/Dicaprate, PEG-4 Laurate, PEG-14M, Styrene/Butadiene Copolymer, Squalane, Citric Acid, PPG-1 Trideceth-6, lodopropynyl Butylcarbamate, Phosphatidylcholine, Cocos Nucifera (Coconut) Liquid Endosperm, Arginine HCL, Laurdimonium Hydroxypropyl Hydrolyzed Keratin, Rosa Canina Fruit Oil, Hydrolyzed Algin, Maris Aqua (Sea Water//Eau De Mer), Sodium Benzoate, Potassium Sorbate, Chlorella Vulgaris Extract, Sodium Hydroxide, Linalool, Limonene, Blue 1 (CI 42090), Ext. Violet 2 (CI 60730), Yellow 5 (CI 19140).

### Dry Repeated Grooming Testing

The composition examples were used in a dry repeated grooming testing regiment to study the effects of hair fiber strengthening. The hair used for the testing were hair tresses of European medium brown color, curl type 1/straight, a size of 8 in. long and 1 in. wide, and were approximately 3g in weight. The hair tresses were also purposely bleached with a 9% hydrogen peroxide solution cause some damage to the hair fibers, in order to better analyze the hair fiber strengthening effects. Before testing, the prepared hair tresses were also cleansed with a 15% sodium laureth sulfate in water to standardize prior to treatment, before being dried.

### Hair Tress Treatment

1. Apply and massage the composition example onto the dry hair tress at a dosage rate of 10% by weight, based on the weight of the tress, and let settle for 20 minutes at room temperature. For the control experiment, a composition example was not applied before shampooing.
2. Dampen the tress under running water at approximately 40° C. and gently squeeze out excess water.
3. Apply and massage a non-conditioning shampoo to the tress for 30 seconds, at a dosage rate of 10% by weight, based on the weight of the tress. The non-conditioning shampoo is Suave® Clarifying Shampoo, containing an ingredient list of:
   Water, Sodium C12-13 Pareth Sulfate, Cocamidopropyl Betaine, Sodium Chloride, Sodium Benzoate, Citric Acid, Fragrance, Hydroxypropyl Methylcellulose, Disodium EDTA, PPG-9, Tocopheryl Acetate, Hexyl Cinnamal, Linalool, Blue 1 (CI42090), Red 33 (CI17200).
5. Rinse the tress under running water at approximately 40° C. for 1 minute and squeeze out excess water.
6. Let the tresses air dry and equilibrate for at least 12 hours.

### Testing

After the tresses have been treated as outlined above, they are groomed using an automatic comber for a total of 3,000 cycles. Results from repeated grooming testing at 3,000 brush strokes are collected. The process was repeated a total of 10 times, and an average result of hair breakages was calculated. The control experiment mentioned above resulted in a mean of 54 hair fiber breakages after 3,000 brush strokes.

The % hair breakage reduction and hair fiber times stronger values are calculated as shown below. The results over the several example compositions are shown further below in Table 3.

$$\% Reduction = ( \frac{\begin{matrix}Average \# Broken Fibers of Control - \\ Average \# Broken Fibers of Example\end{matrix}}{Average \# Broken Fibers of Control} ) \times 100 \%$$

$$Times Stronger = \frac{Average \# Broken Fibers of Control}{Average \# Broken Fibers of Example}$$

TABLE 3
Dry Repeated Grooming Results

| Example # | % Reduction | Times Stronger |
|---|---|---|
| Comp. Ex. 1 | 22% | 1.2 |
| Comp. Ex. 2 | 27% | 1.3 |
| Comp. Ex. 3 | 32% | 1.4 |
| Ex. 1 | 26% | 1.3 |
| Ex. 2 | 39% | 1.6 |
| Ex. 3 | 29% | 1.3 |
| Ex. 4 | 74% | 3.9 |
| Ex. 5 | 76% | 4.1 |
| Ex. 6 | 68% | 3.1 |
| Ex. 7 | 74% | 3.9 |
| Ex. 8 | 46% | 1.8 |
| Ex. 9 | 85% | 6.7 |
| Ex. 10 | 63% | 2.7 |
| Ex. 11 | 69% | 3.1 |
| Ex. 12 | 62% | 2.7 |

As it can be seen from the results, the comparative examples showed some minor benefits of hair fiber strengthening, but were only about 1.2 to 1.4 times stronger than the control experiment, where no pre-shampoo was used. It was surprisingly noted that comparative example 1, which was an anhydrous oil-based pre-shampoo, performed about the same as the emulsion pre-shampoo and conditioner of comparative examples 2 and 3, respectively. However, it is suspected that the lack of a hair fiber strengthening active ingredient resulted in the comparatively minor improvement in hair fiber strengthening when compared to the hair fiber strengthening compositions according to the invention. While a hair fiber strengthening result of 1.2-1.4 times stronger than the control experiment is beneficial, for the purposes of this invention, a result of 2.0 times stronger than the control experiment is desired in order to have a significant enough effect for the consumer to account for the extra difficulty of adding an extra step of applying pre-shampoo into their haircare routine.

The testing results further show that the inclusion of a hair fiber strengthening active ingredient is essential to obtaining increased hair strengthening benefits. A comparison of Examples 4 and 10 show that when the hair fiber strengthening active ingredient is removed, the hair strengthening benefit decreases substantially from 3.9 to 2.7 times stronger than the control.

The additional ingredients of the film former, octyldodecyl citrate crosspolymer, and additional active ingredient oils do not appear to provide better hair strengthening benefits, when reviewing Examples 10 and 11. Example 11 also had a slight decrease in amount of emollients when compared to Example 10, which may have contributed to the minor increase in hair strengthening benefits. Indeed, Example 9 which removes both emollients of caprylic/capric triglyceride and polyglyceryl-10 pentaoleate, leaving only 0.25% of squalane by weight, resulted in the highest hair strengthening benefit of 6.7 times stronger than the control.

Example 8 shows that inclusion of conditioners is also important to obtaining increased hair strengthening benefits. Example 8 decreased the amount of conditioners, and resulted in lower hair strengthening results when compared to other inventive examples. Additionally, the results from Examples 1-3, when compared to the other inventive examples, indicate that stearamidopropyl dimethylamine, a common conditioner, may not provide the hair strengthening effects needed in this type of pre-shampoo composition, because it is easy to rinse out during shampooing. Additionally, Examples 1-3 and 8 when compared to the other inventive examples appear to indicate that behentrimonium chloride should be included in an amount greater than 0.40% by weight, preferably at least 0.75% by weight.

It is also suspected that the identity of the specific humectant may not affect the hair fiber strengthening effect, unless there is a negative interaction between the specific humectant and one of the other ingredients in the hair fiber strengthening composition. More specifically, it appears that there is no effect of switching between butylene glycol, glycerin, and a mixture thereof. However, it has been surprisingly found from Ex. 12 that a high level of humectants will reduce the hair strengthening effects. It is suspected that an ideal level of humectants in the pre-shampoo composition is no higher than 30% by weight, and preferably no higher than 25% by weight.

The hair fiber strengthening compositions described herein have therefore been demonstrated to be useful for increasing the hair fiber strength when used as a pre-shampoo. The benefits are particularly noteworthy when compared to other pre-shampoo products on the market which purport to strengthen hair.

While particular hair fiber strengthening compositions of the invention have been described above, those skilled in the art would understand that various modifications can be made without departing from the spirit and scope of the invention as described above.

1. A haircare composition, comprising:
at least one hair fiber strengthening active ingredient,
at least one humectant,
at least one emollient,
at least one conditioner, and
water.

2. The haircare composition according to claim 1, wherein the haircare composition is a pre-shampoo composition.

3. The haircare composition according to claim 1, wherein the at least one hair fiber strengthening active ingredient comprises hydroxypropylgluconamide and hydroxypropylammonium gluconate.

4. The haircare composition according to claim 1, wherein the haircare composition comprises the at least one hair fiber strengthening active ingredient in an amount of 2%-6% by weight, based on a total weight of the haircare composition.

5. The haircare composition according to claim 1, wherein the at least one humectant is butylene glycol, glycerin, or a mixture thereof.

6. The haircare composition according to claim 1, wherein the haircare composition contains the at least one humectant in an amount less than 30% by weight, based on a total weight of the haircare composition.

7. The haircare composition according to claim 1, wherein the at least one conditioner comprises behentrimonium chloride.

8. The haircare composition according to claim 7, wherein the haircare composition comprises the behentrimonium chloride in an amount of 0.75% by weight or more, based on a total weight of the haircare composition.

9. The haircare composition according to claim 1, wherein the haircare composition does not contain stearamidopropyl dimethylamine.

10. The haircare composition according to claim 1, wherein the at least one emollient comprises squalane.

11. The haircare composition according to claim 1, wherein the haircare composition contains the squalane in an amount of 0.25% by weight, based on a total weight of the haircare composition.

12. The haircare composition according to claim 1, wherein the haircare composition further comprises at least one structurant.

13. The haircare composition according to claim 12, wherein the at least one structurant is cetearyl alcohol, behenyl alcohol, or a mixture thereof.

14. A method of treating hair, comprising:
applying the haircare composition according to claim 1 to the hair, and letting the haircare composition remain on the hair for at least 5 minutes.

15. The method of claim 14, further comprising:
applying a shampoo composition to the hair and rinsing the hair thereafter.
